(19) United States
(12) Patent Application Publication
(10) Pub. No.: US 2022/0389397 A1
(43) Pub. Date: Dec. 8, 2022

(54) DEOXYRIBONUCLEASE VARIANTS AND USES THEREOF

(71) Applicant: THERMO FISHER SCIENTIFIC BALTICS UAB, Vilnius (LI)

(72) Inventor: Gediminas ALZBUTAS, Vilnius (LT)

(21) Appl. No.: 17/775,253

(22) PCT Filed: Nov. 7, 2020
(86) PCT No.: PCT/EP2020/081402

§ 371 Date: May 6, 2022

Related U.S. Application Data
(60) Provisional application No. 62/933,215, filed on Nov. 8, 2019.
(60) Provisional application No. 62/936,977, filed on Nov. 18, 2019.

(51) Int. Cl. C12N 9/22; C12Q 1/6806

(52) U.S. Cl. CPC C12N 9/22; C12Y 301/21001; C12Q 1/6806

(57) ABSTRACT

The present disclosure provides deoxyribonucleases that are salt-tolerant and/or thermolabile. In particular, the present disclosure provides mutant variants of bovine deoxyribonuclease I. Also provided are uses of mutant variants of deoxyribonuclease in various applications where DNA removal is desired and kits containing the same.

### SEQUENCE LISTING

The instant application contains a Sequence Listing which has been submitted electronically in ASCII format and is hereby incorporated by reference in its entirety. Said ASCII copy, created on Nov. 6, 2020, is named LT01511PCT_SL.txt and is 106,166 bytes in size.

### FIELD OF THE INVENTION

The present disclosure provides deoxyribonucleases that are salt-tolerant and/or thermolabile. In particular, the present disclosure provides mutant variants of bovine deoxyribonuclease I. Also provided are uses of mutant variants of deoxyribonuclease and kits containing the same.

### BACKGROUND OF THE INVENTION

Bovine pancreatic deoxyribonuclease I (DNase I) is an enzyme with a wide range of applications. In the field of molecular biology and nucleic acid biochemistry, bovine pancreatic deoxyribonuclease I is used in applications such as nick translation, the production of random DNA fragments, deoxyribonuclease I protection assays such as transcription factor footprinting, removal of DNA template after in vitro transcription, removal of DNA (decontamination or sanitization) from buffers and DNA polymerase enzyme preparations to be used in highly sensitive PCR applications, removal of DNA from RNA samples prior to applications such as RT-PCR, and removal of DNA from other preparations generated by biological and/or biochemical procedures, to name but a few (Sambrook, Fritsch & Maniatis, Molecular Cloning, A Laboratory Manual, 3rd edition, CSHL Press, 2001).

One of the main disadvantages of wild type bovine DNase I limiting its application in molecular biology manipulations is its low tolerance of ionic strength. For example, the use of DNase I to degrade residual genomic DNA in crude cell lysates in RNA sample preparation workflow is often not possible or requires much higher DNase I concentrations—for example, when the salt concentration in the reaction mixture is equivalent to 50 mM NaCl, about 10 times more DNase I needs to be added to achieve efficient DNA digestion. However, when increased enzyme amounts are used, the thermal inactivation of the DNase I requires extended inactivation time or higher temperature of inactivation, also, portion of the enzyme may renature and reactivate when the temperature is lowered. As an alternative one can perform DNase I treatment of isolated RNA sample, but this step requires subsequent DNase I inactivation/removal thereby introducing additional manipulation steps and increased hands-on time. Salt-tolerant DNase would be also useful in decontamination of reagents (buffers with varying concentration of salts, or even solutions containing amplification enzymes) prior to various amplification reactions such PCR or Whole genome amplification (WGA) where no-template control (NTC) is essential.

Protocols for RNA isolation that use DNase I usually require that the enzyme be inactivated or removed prior to downstream reactions. Common approaches include phenol:chloroform extraction followed by the ethanol precipitation, use of DNase removal reagents, or thermal denaturation. Extraction or DNase removal, however, is tedious, requires extensive sample handling and can result in the loss of RNA yield after precipitation. During thermal inactivation the reaction mixture comprising a DNase is heated to a temperature for a period of time that substantially inactivates (e.g., reduces activity by at least 90%, 95%, 99% or more, e.g., 100%) the DNase. For example, the reaction mixture is heated to at least 75° C., and up to 95° C., for 10-20 min. Thermal inactivation, though simple to perform, can jeopardize the integrity of the RNA if performed in the presence of divalent ions. Efficient thermal inactivation of a wild type bovine DNase I that would not affect the quality of RNA requires chelating agent Ethylenediaminetetraacetic acid (EDTA), which, however, may limit the use of the resulting mixture in certain downstream applications that use divalent cations. Thermolabile DNase would be also useful in decontamination of reagents prior to PCR or other amplification reactions such as Whole genome amplification (WGA). For example, in case of PCR reagents, the DNase inactivation step is of concern since high temperature is likely to activate prematurely the hot-start Taq DNA polymerases, thereby increasing primer-dimer formation.

As an alternative to bovine DNase I, a thermolabile DNase from shrimp (dsDNase) can be used to eliminate traces of contaminant DNA. However, it is also salt sensitive, therefore, a lot of enzyme buffers containing salt cannot be subjected to removal of DNA using the dsDNase.

Therefore, there is a need in the art for DNase enzymes having bovine DNase I activity that remain active under high ionic strength conditions and/or can be inactivated at moderate temperature.

### SUMMARY OF THE INVENTION

The present specification describes deoxyribonucleases that are mutant variants of bovine deoxyribonuclease I. In particular, deoxyribonucleases of the current disclosure are thermolabile and/or salt-tolerant. Also provided are uses of deoxyribonucleases and compositions and kits containing deoxyribonucleases.

In some embodiments, a deoxyribonuclease is provided that comprises one or more substitutions at the following selected amino acid positions T14, H44, S75, G105, I130, S138, S174, T177, P197, T205, P227 corresponding to SEQ ID NO: 1, and has at least 80%, 85%, 90%, 95%, 98% or 99% sequence identity with SEQ ID NO: 1. In some embodiments, a deoxyribonuclease has at least 25% of the activity of a deoxyribonuclease having SEQ ID NO: 1 at 23° C. in a buffer comprising 0 mM NaCl. In some embodiments, a deoxyribonuclease maintains 30% or less activity after treatment at 70° C. for 20 minutes. In some embodiments, a deoxyribonuclease maintains at least 30% activity in a buffer comprising 100 mM NaCl.

In some embodiments, a deoxyribonuclease comprises one or more substitutions selected from T14K, T14R, H44R, H44K, S75K, S75R, G105R, G105K, I130L, I130V, I130M, S138K, S138R, S174K, S174R, T177R, T177K, P197S, T205R, I205K, P227S.

In some embodiments, a deoxyribonuclease comprises one or more substitutions selected from G105, I130, S174, T177, P197, T205, P227. In some embodiments, a deoxyribonuclease comprises one or more substitutions selected from G105R, G105K, I130L, I130V, I130M, S174K, S174R, T177R, T177K, P197S, T205R, I205K, P227S. In some embodiments, a deoxyribonuclease comprises one or more substitutions selected from G105R, I130L, S174K, S174R, T177R, P197S, T205R, P227S.

In some embodiments, a deoxyribonuclease comprises one or more substitutions selected from G105R, I130L, P197S, T205R, P227S.

In some embodiments, a deoxyribonuclease comprises substitutions I130L, P197S and T205R. In some embodiments, a deoxyribonuclease comprises substitutions G105R, I130L, P197S, T205R and P227S.

In some embodiments, a deoxyribonuclease comprises one or more substitutions selected from T14, H44, S75, S138, S174, T177. In some embodiments, a deoxyribonuclease comprises one or more substitutions selected from T14K, T14R, H44R, H44K, S75K, S75R, S138K, S138R, S174K, S174R, T177R, T177K. In some embodiments, a deoxyribonuclease comprises substitutions S75K, S138K, S174K. In some embodiments, a deoxyribonuclease further comprises one or more substitutions selected from T14K, H44R, G105R, P197S, P227S.

In some embodiments, a deoxyribonuclease comprises a combination of substitutions selected from: a) H44R, S75K, G105R, I130L, S138K, S174R, P197S, P227S, b) H44R, S75K, G105R, I130L, S138K, S174K, P197S, I205R, P227S, c) H44R, S75K, G105R, I130L, S138K, S174K, P197S, P227S, d) T14K, S75K, G105R, I130L, S138K, S174K, P197S, I205R, or e) T14K, S75K, G105R, I130L, S138K, S174K, P197S, P227S.

In some embodiments, a deoxyribonuclease has an amino acid sequence comprising SEQ ID NO: 37, SEQ ID NO: 38, SEQ ID NO: 39, SEQ ID NO: 40 or SEQ ID NO: 41.

In some embodiments, a deoxyribonuclease comprises a heterologous amino acid sequence. In some embodiments, a heterologous amino acid sequence comprises a sequence-nonspecific double-stranded DNA binding domain. In some embodiments, a DNA binding domain comprises at least one helix-hairpin-helix motif. In some embodiments, a heterologous amino acid sequence comprise a ComEA protein helix-hairpin-helix sequence. In some embodiments, a ComEA protein helix-hairpin-helix sequence is from an organism of a genus selected from Bacillus, Thioalkalivibrio or Halomonas.

In some embodiments, a composition is provided that comprises a deoxyribonuclease and a buffer. In some embodiments, a buffer comprises at least one of Tris-HCl, CaCl₂), MgCl₂ and glycerol.

In some embodiments, a kit for removing DNA from a sample is provided that comprises a deoxyribonuclease and a reaction buffer. In some embodiments, a reaction buffer comprises at least 50 mM or at least 100 mM NaCl.

In some embodiments, use of a deoxyribonuclease to digest DNA in a sample is disclosed. In some embodiments, use of a kit comprising a deoxyribonuclease and a reaction buffer to digest DNA in a sample is disclosed.

In some embodiments, a sample comprises RNA.

In some embodiments, a method for removing DNA from a sample comprising contacting a sample with the deoxyribonuclease under conditions that allow the deoxyribonuclease to digest the DNA is disclosed. In some embodiments, conditions that allow the deoxyribonuclease to digest the DNA include from 50 mM to 600 mM NaCl. In some embodiments, a sample comprises RNA.

### BRIEF DESCRIPTION OF THE DRAWINGS

FIG. 1 shows tolerance to higher salt concentration of DNase I mutant #V in comparison to the wild type DNase I. The reaction was conducted at 37° C. for 30 min in the presence of herring sperm DNA and sodium chloride was added at different concentrations (0-1 M of NaCl). Reaction conditions: 40 mM Tris-HCl pH 8.0, 10 mM MgCl₂, 1 mM CaCl₂).

FIGS. 2A-2C show higher salt tolerance of DNase I mutant #V and DNase I mutant #V-ComEA in comparison to wild type DNase I, when incubated with plasmid DNA. The reaction was performed in the presence of 1 μg of plasmid DNA and samples were incubated at 37° C. for 10 min. “L” represents GeneRuler DNA Ladder Mix (SM0331, Thermo Fisher Scientific), “C-” is undigested control pUC19 DNA (the main band represents supercoiled form). In the lane of 0.4 M NaCl in the wild type DNase I electrophoretic gel picture linear (lower band) and nicked (upper band) forms of the plasmid can be observed; the same applies to the rest of the lanes in all pictures. An observed smear of DNA represents DNA digestion by DNase I to various extent.

FIG. 3 shows thermolability of DNase I mutant #V as compared to wild-type DNase I. The thermolability experiments were conducted in the presence of DNase I and DNase I mutant #V which were incubated for 30 min at different temperatures (4-95° C.) before conducting the DNase assay. The DNase assay was conducted at 37° C. for 30 min in the presence of herring sperm DNA. Reaction conditions: 40 mM Tris-HCl pH 8.0, 10 mM MgCl₂, 1 mM CaCl₂.

FIG. 4 shows thermolability of DNase I mutant #V as compared to wild-type DNase I. Samples were incubated for 30 min at different temperatures (4-95° C.) before conducting the DNase assay. The DNA assay was then performed in the presence of 1 μg of plasmid DNA and samples were incubated at 37° C. for 10 min. The reaction was stopped by adding 6×DNA Loading Dye and SDS Solution and inactivated by heating at 70° C. for 5 min. Samples were run on 1% agarose gels. The leftmost lane is GeneRuler DNA Ladder Mix (SM0331, Thermo Fisher Scientific), Lane “C-” -template DNA without DNase I; Lane “C+”-template DNA with DNase I added (pre-incubation at 4° C.); 50, 60, 70, 80, 95 denote the temperatures in ° C. at which the DNase I was incubated before performing the DNA assay.

FIGS. 5A-5B shows the results of sanitization (removal of DNA from reagents) reaction prior to Whole genome amplification reaction (WGA). a) part shows the scheme of experiment. b) part shows the photos of 1, 3 and 10 HeLa cells captured using Laser capture microdissection (LCM). The lower part of b) shows the results of WGA reaction. “M”-FastRuler High Range DNA Ladder (#SM1123, Thermo Fisher Scientific), “ntc”-no-template control, numbers above the lanes indicate number of cells used in cell lysates.

### DESCRIPTION OF THE SEQUENCES

A listing of certain sequences referenced herein is provided.

Description of the Sequences and SEQ ID Nos

| Description | Sequence | # |
|---|---|---|
| Wt bovine | LKIAAFNIRT FGETKMSNAT LASYIVRIVR RYDIVLIQEV | 1 |
| DNase I | RDSHLVAVGK LLDYLNQDDP NTYHYVVSEP LGRNSYKERY | |
| | LFLFRPNKVS VLDTYQYDDG CESCGNDSFS REPAVVKFSS | |
| | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| His-wtDNase I | MAGSHHHHHH GMASMTGGQQ MGRSGDDDDK LKIAAFNIRT | 2 |
| | FGETKMSNAT LASYIVRIVR RYDIVLIQEV RDSHLVAVGK | |
| | LLDYLNQDDP NTYHYVVSEP LGRNSYKERY LFLFRPNKVS | |
| | VLDTYQYDDG CESCGNDSFS REPAVVKFSS HSTKVKEFAI | |
| | VALHSAPSDA VAEINSLYDV YLDVQQKWHL NDVMLMGDFN | |
| | ADCSYVTSSQ WSSIRLRTSS TFQWLIPDSA DTTATSTNCA | |
| | YDRIVVAGSL LQSSVVPGSA APFDFQAAYG LSNEMALAIS | |
| | DHYPVEVTLT | |
| DNase I | LKIAAFNIRT FGEXKMSNAT LASYIVRIVR RYDIVLIQEV | 3 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| T14X | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT; | |
| | wherein X is selected from K, R, Q, N and H. | |
| DNase I | LKIAAFNIRT FGEKKMSNAT LASYIVRIVR RYDIVLIQEV | 4 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| T14K | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGERKMSNAT LASYIVRIVR RYDIVLIQEV | 5 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| T14R | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 6 |
| mutant with a | RDSXLVAVGK LLDYLNQDDP NTYHYVVSEP LGRNSYKERY | |
| substitution | LFLFRPNKVS VLDTYQYDDG CESCGNDSFS REPAVVKFSS | |
| H44X | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT; | |
| | wherein X is selected from K, R, Q and N. | |
| DNase I | LKIAAFNIRT FGETKMSNAT LASYIVRIVR RYDIVLIQEV | 7 |
| mutant with a | RDSRLVAVGK LLDYLNQDDP NTYHYVVSEP LGRNSYKERY | |
| substitution | LFLFRPNKVS VLDTYQYDDG CESCGNDSFS REPAVVKFSS | |
| H44R | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 8 |
| mutant with a | RDSKLVAVGK LLDYLNQDDP NTYHYVVSEP LGRNSYKERY | |
| substitution | LFLFRPNKVS VLDTYQYDDG CESCGNDSFS REPAVVKFSS | |
| H44K | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 9 |
| mutant with a | RDSHLVAVGK LLDYLNQDDP NTYHYVVSEP LGRNXYKERY | |
| substitution | LFLFRPNKVS VLDTYQYDDG CESCGNDSFS REPAVVKFSS | |
| S75X | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT; | |
| | wherein X is selected from K, R, Q, N and H. | |
| DNase I | LKIAAFNIRT FGETKMSNAT LASYIVRIVR RYDIVLIQEV | 10 |
| mutant with a | RDSHLVAVGK LLDYLNQDDP NTYHYVVSEP LGRNKYKERY | |
| substitution | LFLFRPNKVS VLDTYQYDDG CESCGNDSFS REPAVVKFSS | |
| S75K | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 11 |
| mutant with a | RDSHLVAVGK LLDYLNQDDP NTYHYVVSEP LGRNRYKERY | |
| substitution | LFLFRPNKVS VLDTYQYDDG CESCGNDSFS REPAVVKFSS | |
| S75R | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 12 |
| mutant with a | RDSHLVAVGK LLDYLNQDDP NTYHYVVSEP LGRNSYKERY | |
| substitution | LFLFRPNKVS VLDTYQYDDG CESCXNDSFS REPAVVKFSS | |
| G105X | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT; | |
| | wherein X is selected from R, K, Q, N and H. | |
| DNase I | LKIAAFNIRT FGETKMSNAT LASYIVRIVR RYDIVLIQEV | 13 |
| mutant with a | RDSHLVAVGK LLDYLNQDDP NTYHYVVSEP LGRNSYKERY | |
| substitution | LFLFRPNKVS VLDTYQYDDG CESCRNDSFS REPAVVKFSS | |
| G105R | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 14 |
| mutant with a | RDSHLVAVGK LLDYLNQDDP NTYHYVVSEP LGRNSYKERY | |
| substitution | LFLFRPNKVS VLDTYQYDDG CESCRNDSFS REPAVVKFSS | |
| G105K | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 15 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| I130X | HSTKVKEFAX VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT; | |
| | wherein X is selected from M, V and L. | |
| DNase I | LKIAAFNIRT FGETKMSNAT LASYIVRIVR RYDIVLIQEV | 16 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| I130L | HSTKVKEFAL VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 17 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| S138X | HSTKVKEFAI VALHSAPXDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT; | |
| | wherein X is selected from K, R, Q, N and H. | |
| DNase I | LKIAAFNIRT FGETKMSNAT LASYIVRIVR RYDIVLIQEV | 18 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| S138K | HSTKVKEFAI VALHSAPKDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 19 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| S138R | HSTKVKEFAI VALHSAPRDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 20 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| S174X | HSTKVKEFAI VALHSAPSDA VAEINSLYDV YLDVQQKWHL | |
| | NDVMLMGDFN ADCXYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT; | |
| | wherein X is selected from K, R, Q, N and H. | |
| DNase I | LKIAAFNIRT FGETKMSNAT LASYIVRIVR RYDIVLIQEV | 21 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| S174K | HSTKVKEFAI VALHSAPSDA VAEINSLYDV YLDVQQKWHL | |
| | NDVMLMGDFN ADCKYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 22 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| S174R | HSTKVKEFAI VALHSAPSDA VAEINSLYDV YLDVQQKWHL | |
| | NDVMLMGDFN ADCRYVTSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 23 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| T177X | HSTKVKEFAI VALHSAPSDA VAEINSLYDV YLDVQQKWHL | |
| | NDVMLMGDFN ADCSYVXSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT; | |
| | wherein X is selected from R, K, Q, N and H. | |
| DNase I | LKIAAFNIRT FGETKMSNAT LASYIVRIVR RYDIVLIQEV | 24 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| T177R | HSTKVKEFAI VALHSAPSDA VAEINSLYDV YLDVQQKWHL | |
| | NDVMLMGDFN ADCSYVRSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 25 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| T177K | HSTKVKEFAI VALHSAPSDA VAEINSLYDV YLDVQQKWHL | |
| | NDVMLMGDFN ADCSYVKSSQ WSSIRLRTSS TFQWLIPDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 26 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| P197X | HSTKVKEFAI VALHSAPSDA VAEINSLYDV YLDVQQKWHL | |
| | NDVMLMGDFN ADCSYVTSSQ WSSIRLRTSS TFQWLIXDSA | |
| | DTTATSTNCA YDRIVVAGSL LQSSVVPGSA APFDFQAAYG | |
| | LSNEMALAIS DHYPVEVTLT; | |
| | wherein X is selected from S, R, N, D, C, E, | |
| | Q, G, H, I, L, K, M, F, P, T, W, Y, V. | |
| DNase I | LKIAAFNIRT FGETKMSNAT LASYIVRIVR RYDIVLIQEV | 27 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| P197S | HSTKVKEFAI VALHSAPSDA VAEINSLYDV YLDVQQKWHL | |
| | NDVMLMGDFN ADCSYVTSSQ WSSIRLRTSS TFQWLISDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 28 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| T205X | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTAXSTNCA YDRIVVAGSL LQSSVVPGSA APFDFQAAYG | |
| | LSNEMALAIS DHYPVEVTLT; | |
| | wherein X is selected from R, K, Q, N and H. | |
| DNase I | LKIAAFNIRT FGETKMSNAT LASYIVRIVR RYDIVLIQEV | 29 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| T205R | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTARSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 30 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| T205K | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTAKSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 31 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| P227X | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVXGSA APFDFQAAYG | |
| | LSNEMALAIS DHYPVEVTLT; | |
| | wherein X is selected from S, R, N, D, C, E, | |
| | Q, G, H, I, L, K, M, F, P, T, W, Y, V. | |
| DNase I | LKIAAFNIRT FGETKMSNAT LASYIVRIVR RYDIVLIQEV | 32 |
| mutant with a | RDSHLVAVGK LLDYLNQDDP NTYHYVVSEP LGRNSYKERY | |
| substitution | LFLFRPNKVS VLDTYQYDDG CESCGNDSFS REPAVVKFSS | |
| P227S | HSTKVKEFAI VALHSAPSDA VAEINSLYDV YLDVQQKWHL | |
| | NDVMLMGDFN ADCSYVTSSQ WSSIRLRTSS TFQWLIPDSA | |
| | DTTATSTNCA YDRIVVAGSL LQSSVVSGSA APFDFQAAYG | |
| | LSNEMALAIS DHYPVEVTLT | |
| DNase I | LKIAAFNIRT FGETKMSNAT LASYIVRIVR RYDIVLIQEV | 33 |
| mutant Muti 6 | RDSHLVAVGK LLDYLNQDDP NTYHYVVSEP LGRNSYKERY | |
| | LFLFRPNKVS VLDTYQYDDG CESCGNDSFS REPAVVKFSS | |
| | HSTKVKEFAL VALHSAPSDA VAEINSLYDV YLDVQQKWHL | |
| | NDVMLMGDFN ADCSYVTSSQ WSSIRLRTSS TFQWLISDSA | |
| | DTTARSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 34 |
| mutant Muti 8 | RDSHLVAVGK LLDYLNQDDP NTYHYVVSEP LGRNSYKERY | |
| | LFLFRPNKVS VLDTYQYDDG CESCRNDSFS REPAVVKFSS | |
| | HSTKVKEFAL VALHSAPSDA VAEINSLYDV YLDVQQKWHL | |
| | NDVMLMGDFN ADCSYVTSSQ WSSIRLRTSS TFQWLISDSA | |
| | DTTATSTNCA YDRIVVAGSL LQSSVVSGSA APFDFQAAYG | |
| | LSNEMALAIS DHYPVEVTLT | |
| DNase I | LKIAAFNIRT FGEKKMSNAT LASYIVRIVR RYDIVLIQEV | 35 |
| mutant Mut4 | RDSHLVAVGK LLDYLNQDDP NTYHYVVSEP LGRNKYKERY | |
| | LFLFRPNKVS VLDTYQYDDG CESCRNDSFS REPAVVKFSS | |
| | HSTKVKEFAI VALHSAPKDA VAEINSLYDV YLDVQQKWHL | |
| | NDVMLMGDFN ADCKYVTSSQ WSSIRLRTSS TFQWLISDSA | |
| | DTTATSTNCA YDRIVVAGSL LQSSVVPGSA APFDFQAAYG | |
| | LSNEMALAIS DHYPVEVTLT | |
| DNase I | LKIAAFNIRT FGETKMSNAT LASYIVRIVR RYDIVLIQEV | 36 |
| mutant Muti 3 | RDSRLVAVGK LLDYLNQDDP NTYHYVVSEP LGRNKYKERY | |
| | LFLFRPNKVS VLDTYQYDDG CESCRNDSFS REPAVVKFSS | |
| | HSTKVKEFAI VALHSAPKDA VAEINSLYDV YLDVQQKWHL | |
| | NDVMLMGDFN ADCKYVTSSQ WSSIRLRTSS TFQWLISDSA | |
| | DTTATSTNCA YDRIVVAGSL LQSSVVSGSA APFDFQAAYG | |
| | LSNEMALAIS DHYPVEVTLT | |
| DNase I | LKIAAFNIRT FGEKKMSNAT LASYIVRIVR RYDIVLIQEV | 37 |
| mutant #I | RDSHLVAVGK LLDYLNQDDP NTYHYVVSEP LGRNKYKERY | |
| | LFLFRPNKVS VLDTYQYDDG CESCRNDSFS REPAVVKFSS | |
| | HSTKVKEFAL VALHSAPKDA VAEINSLYDV YLDVQQKWHL | |
| | NDVMLMGDFN ADCKYVTSSQ WSSIRLRTSS TFQWLISDSA | |
| | DTTARSTNCA YDRIVVAGSL LQSSVVPGSA APFDFQAAYG | |
| | LSNEMALAIS DHYPVEVTLT | |
| DNase I | LKIAAFNIRT FGEKKMSNAT LASYIVRIVR RYDIVLIQEV | 38 |
| mutant #II | RDSHLVAVGK LLDYLNQDDP NTYHYVVSEP LGRNKYKERY | |
| | LFLFRPNKVS VLDTYQYDDG CESCRNDSFS REPAVVKFSS | |
| | HSTKVKEFAL VALHSAPKDA VAEINSLYDV YLDVQQKWHL | |
| | NDVMLMGDFN ADCKYVTSSQ WSSIRLRTSS TFQWLISDSA | |
| | DTTATSTNCA YDRIVVAGSL LQSSVVSGSA APFDFQAAYG | |
| | LSNEMALAIS DHYPVEVTLT | |
| DNase I | LKIAAFNIRT FGETKMSNAT LASYIVRIVR RYDIVLIQEV | 39 |
| mutant #III | RDSRLVAVGK LLDYLNQDDP NTYHYVVSEP LGRNKYKERY | |
| | LFLFRPNKVS VLDTYQYDDG CESCRNDSFS REPAVVKFSS | |
| | HSTKVKEFAL VALHSAPKDA VAEINSLYDV YLDVQQKWHL | |
| | NDVMLMGDFN ADCKYVTSSQ WSSIRLRTSS TFQWLISDSA | |
| | DTTARSTNCA YDRIVVAGSL LQSSVVSGSA APFDFQAAYG | |
| | LSNEMALAIS DHYPVEVTLT | |
| DNase I | LKIAAFNIRT FGETKMSNAT LASYIVRIVR RYDIVLIQEV | 40 |
| mutant #IV | RDSRLVAVGK LLDYLNQDDP NTYHYVVSEP LGRNKYKERY | |
| | LFLFRPNKVS VLDTYQYDDG CESCRNDSFS REPAVVKFSS | |
| | HSTKVKEFAL VALHSAPKDA VAEINSLYDV YLDVQQKWHL | |
| | NDVMLMGDFN ADCKYVTSSQ WSSIRLRTSS TFQWLISDSA | |
| | DTTATSTNCA YDRIVVAGSL LQSSVVSGSA APFDFQAAYG | |
| | LSNEMALAIS DHYPVEVTLT | |
| DNase I | LKIAAFNIRT FGETKMSNAT LASYIVRIVR RYDIVLIQEV | 41 |
| mutant #V | RDSRLVAVGK LLDYLNQDDP NTYHYVVSEP LGRNKYKERY | |
| | LFLFRPNKVS VLDTYQYDDG CESCRNDSFS REPAVVKFSS | |
| | HSTKVKEFAL VALHSAPKDA VAEINSLYDV YLDVQQKWHL | |
| | NDVMLMGDFN ADCRYVTSSQ WSSIRLRTSS TFQWLISDSA | |
| | DTTATSTNCA YDRIVVAGSL LQSSVVSGSA APFDFQAAYG | |
| | LSNEMALAIS DHYPVEVTLT | |
| DNase I | LKIAAFNIRT FGETKMSNAT LASYIVRIVR RYDIVLIQEV | 42 |
| mutant #V- | RDSRLVAVGK LLDYLNQDDP NTYHYVVSEP LGRNKYKERY | |
| ComEA | LFLFRPNKVS VLDTYQYDDG CESCRNDSFS REPAVVKFSS | |
| | HSTKVKEFAL VALHSAPKDA VAEINSLYDV YLDVQQKWHL | |
| | NDVMLMGDFN ADCRYVTSSQ WSSIRLRTSS TFQWLISDSA | |
| | DTTATSTNCA YDRIVVAGSL LQSSVVSGSA APFDFQAAYG | |
| | LSNEMALAIS DHYPVEVTLT GEETAVQQGG GGSVQSDGGK | |
| | GALVNINTAT LEELQGISGV GPSKAEAIIA YREENGRFQT | |
| | IEDITKVSGI GEKSFEKIKS SITVK | |
| Top strand of | GTTGGTGGGTTTGGGTGTGGGTTTGTGTTT-BHQ1 | 43 |
| Fluorescent | | |
| DNA duplex, | | |
| labeled with | | |
| 3′-BHQ1 | | |
| Bottom strand | FAM-AAACACAAACCCACACCCAAACCCACCAAC | 44 |
| of Fluorescent | | |
| DNA duplex, | | |
| labeled with | | |
| 5′-FAM | | |
| Forward | TGATGACGGTGAAAACCTCTGA | 45 |
| primer | | |
| Reverse | CGGCATCCGCTTACAGACA | 46 |
| primer | | |

### DETAILED DESCRIPTION

The present disclosure relates to deoxyribonucleases, in particular, mutant variants of bovine DNase I. In some embodiments, the mutant variants of DNase I are salt-tolerant. In some embodiments, the mutant variants of DNase I are thermolabile. In some embodiments, the mutant variants of DNase I are salt-tolerant and thermolabile.

### Definitions

Deoxyribonuclease I (DNase I) is a phosphodiesterase capable of hydrolyzing polydeoxyribonucleic acid. It acts to extensively and non-specifically degrade DNA. As used herein, the terms “bovine DNase I” and “wild-type DNase” interchangeably refer to a polypeptide having the amino acid sequence SEQ ID NO: 1. Accordingly, “mutant variant of DNase” or “mutant DNase” as used herein, refer to a DNase that comprises one or more substitutions, with respect to the amino acid sequence having SEQ ID NO: 1. A “mutant variant of DNase” or “mutant DNase” may further comprise extensions (e.g. by one or more amino acids added at the amino- or carboxyl-terminus) and/or truncations (e.g. by one or more amino acids deleted from the amino- or carboxyl-terminus with respect to the amino acid sequence of SEQ ID NO: 1.

The term “thermolabile DNase” refers to a DNase the enzymatic activity of which is expressed at reduced levels after incubation at a temperature of 60° C., 65° C., and/or 70° C. when compared to the enzymatic activity of a reference DNase (e.g. bovine DNase I) under equal conditions. As used herein, a thermolabile DNase is less resistant to inactivation at said temperatures than a non-thermolabile (e.g. bovine) DNase. Thermolability is measured as the % residual activity of a DNase after incubation for a given time period at given temperature. For example, thermolability of a DNase I can be measured as the % activity after incubation for 20 min at 70° C. in a reaction buffer (10 mM Tris-HCl pH 7.5, 1 mM CaCl₂) as compared to the activity of said DNase I incubated for 20 min at 4° C. under otherwise equal conditions (i.e. a DNase that is incubated at 4° C., is not heated). As an example, a first DNase I that retains 30% of activity (i.e. has 30% residual activity) after incubation for 20 min at 70° C. as compared to its activity when incubated at 4° C. under otherwise equal conditions is considered thermolabile in comparison to a second DNase I that retains more than 30% activity (i.e. has more than 30% residual activity) after incubation for 20 min at 70° C. as compared to its activity when incubated at 4° C. under otherwise equal conditions.

Alternatively, the change in activity of a DNase I after incubation at a temperature of 60° C., 65° C., and/or 70° C. may be calculated using Formula I:

[[(Activity (heated)−Activity (not-heated)]/Activity (not-heated)]×100%;

Wherein Activity (not-heated) is the activity of a DNase after incubation at 4° C., and wherein Activity (heated) is the activity of a DNase after incubation at a defined temperature that is higher than 4° C. (for example, 70° C.). As an example, when Activity (heated) is lower than Activity (not-heated), the calculation will result in a negative percent value, and thus will show a decrease in activity.

The term “salt-tolerant DNase” refers to a DNase that is not inactivated or is inactivated only to a certain extent at elevated salt concentrations in the reaction buffer. Salt tolerance is measured as the % residual activity of a DNase maintained for a given time period at a defined salt concentration in a buffered solution. For example, salt tolerance of a DNase I can be measured as the % activity after incubation in a buffer comprising 100 mM NaCl as compared to the activity of said DNase I under equal conditions but in the presence of 0 mM NaCl. For example, salt tolerance of a DNase I can be measured as the % activity after incubation for 10 minutes at 23° C. in a buffer: 10 mM Tris-HCl pH 7.5, 1 mM CaCl₂), 100 mM NaCl as compared to the activity of said DNase I under the same conditions but in the presence of 0 mM NaCl. Alternatively, salt tolerance of a DNase I can be measured as the % activity after incubation for 30 minutes at 37° C. in a buffer: 10 mM Tris-HCl pH 7.5, 1 mM CaCl₂), 100 mM NaCl as compared to the activity of said DNase I under the same conditions but in the presence of 0 mM NaCl. As an example, a first DNase I that retains 50% of activity (i.e. has 50% residual activity) after incubation for 10 min in a reaction buffer comprising 100 mM NaCl as compared to its activity after incubation at equal conditions, but in the presence of 0 mM NaCl, is considered to have increased salt tolerance in comparison to a second DNase I that retains less than 50% activity after incubation for 10 min in a reaction buffer comprising 100 mM NaCl as compared to its activity after incubation at equal conditions, but in the presence of 0 mM NaCl. In particular, a “salt-tolerant DNase” may have higher activity compared to a wild type DNase (e.g. a bovine DNase I) at NaCl concentrations of 50 mM, 100 mM and more, under otherwise equal conditions. In some aspects, a “salt-tolerant DNase” may have higher activity at 100 mM NaCl concentration as compared to the activity of “salt-tolerant DNase” at 0 mM NaCl concentration in the reaction buffer. In some instances, the activity of a salt-tolerant DNase may be increased by at least 5%, 10%, 20%, 30%, 40%, 50%, 60%, 70%, 80%, 90%, 100%, 110%, 120%, 130%, 140%, 150%, 160%, 170% in a reaction buffer comprising 100 mM NaCl when compared to the activity of the salt-tolerant DNase in the reaction buffer comprising 0 mM NaCl.

Alternatively, the change in activity of a DNase I after incubation in a buffer comprising 100 mM NaCl, as compared to its activity in a buffer comprising 0 mM NaCl, may be calculated using Formula II:

[[(Activity (100 mM)−Activity (0 mM)]/Activity (0 mM)]×100%

Wherein Activity (0 mM) is the activity of a DNase after incubation in a buffer comprising 100 mM NaCl, and wherein Activity (100 mM) is the activity of a DNase after incubation in a buffer comprising 0 mM NaCl. As an example, when Activity (100 mM) is lower than Activity (0 mM), the calculation will result in a negative percent value, and thus will show a decrease in activity. In alternative example, when Activity (100 mM) is higher than Activity (0 mM), the calculation will result in a positive percent value, and thus will show an increase in activity

The terms “identical” or percent “identity,” in the context of two or more polypeptide sequences, refer to two or more sequences or subsequences that have a homology of 100% when aligned. Sequences are “X % identical” if two sequences have a specified X percentage of amino acid residues that are the same (i.e., X % may be 80%, 85%, 90%, or 95% identity over a specified region, or, when not specified, over the entire sequence), when compared and aligned for maximum correspondence over a comparison window, designated region as measured using one of the sequence comparison algorithms or by manual alignment and visual inspection. A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/).

As used herein, a term “sample” refers to any solution that comprises or may comprise DNA. In some aspects, a sample may comprise RNA. In some aspects, a sample may comprise various amounts of DNA in comparison to RNA. In some aspects, a sample may comprise other components, such as salts, detergents, buffer salts, proteins etc.

### II. DNase

The current disclosure relates to a bovine deoxyribonuclease I (DNase I) and mutants derived therefrom. The amino acid sequence of bovine DNase I which is also referred to as wildtype DNase I is SEQ ID NO: 1. In some aspects, the disclosure relates to mutant variants of DNase I that comprise one or more amino acid substitutions compared to the SEQ ID NO: 1. For example, amino acid substitutions identical or similar to those described in more detail below can be introduced to a DNase having SEQ ID NO: 1 or a subsequence thereof. Alternative amino acid substitutions can be made using any of the techniques and guidelines for conservative and non-conservative amino acids as set forth, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. As used herein, “non-conservative substitution” refers to the substitution of an amino acid in one class with an amino acid from another class; for example, substitution of an Ala, a class II residue, with a class III residue such as Asp, Asn, Glu, or Gln. Appropriate amino acid alterations allowed in relevant positions may be confirmed by testing the resulting modified DNases for activity in the in vitro assays known in the art or as described in the Examples below. In some aspects, a mutant DNase I may comprise up to nine substitutions. In some aspects, a mutant DNase I may comprise more than nine substitutions. In some aspects, a mutant DNase I comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1 or a subsequence thereof. In some aspects, a mutant DNase I comprises one or more substitutions at the following selected amino acid positions T14, H44, S75, G105, I130, S138, S174, T177, P197, T205, P227 corresponding to SEQ ID NO: 1, and has at least 80%, 85%, 90%, 95%, 98% or 99% sequence identity with SEQ ID NO: 1.

In some aspects, a mutant DNase I has DNase activity of at least about 25%, 30%, 40%, 50%, 60%, 70%, 80%, 90% or 100% of that of the corresponding wild type DNase I (SEQ ID NO: 1), under equal conditions. In some aspects, the DNase activity of a mutant DNase and wild type DNase I can be measured and compared at 23° C. in a buffer comprising 0 mM NaCl. For example, the activity of a mutant DNase I and wild type DNase I can be measured and compared after incubation for 10 minutes at 23° C. in a reaction buffer: 10 mM Tris-HCl pH 7.5, 1 mM CaCl₂. In some aspects, the DNase activity of a mutant DNase and wild type DNase I can be measured and compared at 23° C. in a buffer comprising 100 mM NaCl. For example, the activity of a mutant DNase I and wild type DNase I can be measured and compared after incubation for 10 minutes at 23° C. in a reaction buffer: 10 mM Tris-HCl pH 7.5, 1 mM CaCl₂, 100 mM NaCl. In some aspects, a mutant DNase I has a DNase activity of at least 90% of that of the corresponding wild type DNase I, at 23° C. in a buffer comprising 100 mM NaCl. In some aspects, a mutant DNase I has a DNase activity of at least 90% of that of the corresponding wild type DNase I, after incubation for 10 minutes at 23° C. in a reaction buffer: 10 mM Tris-HCl pH 7.5, 1 mM CaCl₂), 100 mM NaCl. In some aspects, a mutant DNase I has DNase activity that is more than 100% of the activity of the wild type DNase I, under equal conditions. In some aspects, a mutant DNase I has DNase activity that is more than 100% of the activity of the wild type DNase I, when the activity is measured at 23° C. in a buffer comprising 100 mM NaCl. For example, the activity of a mutant DNase I and wild type DNase I can be measured and compared after incubation for 10 minutes at 23° C. in a reaction buffer: 10 mM Tris-HCl pH 7.5, 1 mM CaCl₂, 100 mM NaCl.

Alternatively, the activity of a mutant DNase I and wild type DNase I can be measured and compared after incubation for 30 minutes at 37° C., in a reaction buffer: 10 mM Tris-HCl pH 7.5, 1 mM CaCl₂. In some aspects, the activity of a mutant DNase I and wild type DNase I can be measured and compared after incubation for 30 minutes at 37° C., in a reaction buffer: 10 mM Tris-HCl pH 7.5, 1 mM CaCl₂, 100 mM NaCl. In some aspects, a mutant DNase I has at least 90% of the activity of the wild type DNase I, after incubation for 30 minutes at 37° C. in a reaction buffer: 10 mM Tris-HCl pH 7.5, 1 mM CaCl₂), 100 mM NaCl. In some aspects, a mutant DNase I has DNase activity that is more than 100% of the activity of the wild type DNase I, when the activity is measured at 37° C. in a buffer comprising 100 mM NaCl. In some aspects, the activity of a mutant DNase I and wild type DNase I can be measured and compared after incubation for 30 minutes at 37° C. in a reaction buffer: 10 mM Tris-HCl pH 7.5, 1 mM CaCl₂), 100 mM NaCl.

### Thermolabile DNase

In some aspects, a thermolabile DNase I comprises at least one substitution as compared to SEQ ID NO:1 in one or more amino acid positions selected from G105, I130, S174, T177, P197, T205, P227. In some aspects, a thermolabile DNase I comprises one or more substitutions at amino acid position selected from G105R, G105K, I130L, I130V, I130M, S174K, S174R, T177R, T177K, P197S, T205R, T205K, P227S. In further aspects, a thermolabile DNase I comprises one or more substitutions at amino acid position selected from G105R, I130L, S174K, S174R, T177R, P197S, T205R, P227S. In some aspects, the activity of a thermolabile DNase after incubation at 70° C. for 20 min in a buffer with 0 mM NaCl may be 25% or less, as compared to the activity of the thermolabile DNase I prior to incubation. In some aspects, the activity of a thermolabile DNase I after incubation for 10 min at 70° C. in a buffer with 0 mM NaCl may be 15% or less, as compared to the activity of the thermolabile DNase I prior to incubation.

In some aspects, at least one substitution of a thermolabile DNase I is selected from the following amino acid substitutions: G105R, I130L, P197S, T205R, P227S, wherein the activity of a thermolabile DNase I after incubation for 10 min at 70° C. in a reaction buffer with 0 mM NaCl is 5% or less, as compared to the activity of the thermolabile DNase I prior to incubation. In some aspects, a thermolabile DNase I comprises the following substitutions: I130L, P197S and T205R; wherein the activity a thermolabile DNase I after incubation for 20 min at 65° C. in a reaction buffer with 0 mM NaCl is 5% or less, as compared to the activity of the thermolabile DNase I prior to incubation. In some aspects, a thermolabile DNase I comprises the following substitutions: G105R, I130L, P197S and P227S; wherein the activity a thermolabile DNase I after incubation for 20 min at 65° C. in a reaction buffer with 0 mM NaCl is 5% or less, as compared to the activity of the thermolabile DNase I prior to incubation. In some aspects, a thermolabile DNase I comprises SEQ ID NO: 33 or SEQ ID NO: 34.

In some aspects, a DNase I comprises an identical number of amino acids as the corresponding wild type DNase I sequence SEQ ID NO: 1. In some aspects, a thermolabile DNase I may further comprise a heterologous amino acid sequence. In some aspects, a heterologous amino acid sequence is a tag sequence at the N-terminal part of a DNase I. In some aspects, a tag sequence my be at the C-terminal part of a DNase I. In some aspects, a tag sequence may be at the N-terminal and C-terminal parts of a DNase I. In some aspects, a tag comprises from 1 to 60 or more amino acids in length. In some aspects, a tag comprises from 5 to 60 amino acids or from 20 to 30 amino acids in length. In some aspects, a DNase I comprising a tag sequence at N and/or C terminal part of a DNase I has increased thermolability as compared to DNase I that does not comprise the tag sequence. In some aspects a tag sequence comprises 6 histidine amino acids (SEQ ID NO: 47) (i.e. is a Histag). In some aspects, a tag sequence comprising a Histag is at the N-terminal part of a DNase I. In some aspects, the activity of a DNase I comprising an N-terminal tag with a Histag sequence after incubation at 70° C. for 20 min in a buffer with 0 mM NaCl may be 30% or less, as compared to the activity of said DNase I prior to incubation. In some aspects, the DNase portion in a DNase I comprising N-terminal tag with a Histag sequence is a mutant DNase I according to the present disclosure. In some aspects, the DNase portion in a DNase I comprising N-terminal tag with a Histag sequence is a wild type DNase I (SEQ ID NO: 1). In some aspects, a DNase I comprising N-terminal tag with a Histag sequence comprises SEQ ID NO: 2.

### Salt-Tolerant DNase

A salt-tolerant DNase I of the present disclosure has a higher activity compared to the bovine DNase I enzyme having SEQ ID NO: 1, at elevated NaCl concentrations, in particular at values in the range of 50 mM to 400 mM NaCl.

Therefore, a salt-tolerant DNase I of the present disclosure may have an activity at 100 mM NaCl, 200 mM NaCl, 300 mM and/or 400 mM NaCl concentration that is greater than that of a wild type DNase I (SEQ IDNO: 1). In some aspects, a salt-tolerant DNase I maintains at least 30% activity in a buffer comprising 100 mM NaCl. In some aspects, a salt-tolerant DNase I maintains at least 30% activity in a buffer comprising 100 mM NaCl as compared to the activity of said DNase I under the same conditions but in the presence of 0 mM NaCl. In some aspects, a salt-tolerant DNase I maintains at least 30% activity for 10 minutes at 23° C. in a buffer comprising 100 mM NaCl for 10 minutes at 23° C. in a buffer: 10 mM Tris-HCl pH 7.5, 1 mM CaCl₂), 100 mM NaCl, as compared to the activity of said DNase I under the same conditions but in the presence of 0 mM NaCl.

In some aspects, a salt-tolerant DNase I comprises at least one substitution at amino acid position corresponding to the (SEQ ID NO:1), wherein one or more amino acid position is selected from T14, H44, S75, S138, S174, T177. In some aspects, a salt-tolerant DNase I comprises at least one substitution at amino acid position selected from T14K, T14R, H44R, H44K, S75K, S75R, S138K, S138R, S174K, S174R, T177R, T177R. In a further aspect, a salt-tolerant DNaseI retains more than 30% of DNase I activity in a reaction buffer containing 100 mM NaCl compared to its activity in a reaction buffer containing 0 mM NaCl. In some aspects, the DNase I mutant retains at least about 35%, 40%, 45%, 50%, 55%, 60%, 65%, 70% of DNase I activity in a reaction buffer containing 100 mM NaCl as compared to its activity in a reaction buffer containing 0 mM NaCl. In some aspects, a salt-tolerant DNase I comprises one or more of the substitutions selected from T14K, H44R, S75K, S138K, S174K. In some aspects, a salt-tolerant DNase I comprises the substitutions H44R, S75K, G105R, S138K, S174K, P197S, P227. In some aspects, a salt-tolerant DNase I comprises T14K, S75K, G105R, S138K, S174K, P197S. In some aspects, a salt-tolerant DNase I comprises SEQ ID NO: 35 or SEQ ID NO: 36.

In some aspects, a salt-tolerant DNase I comprises an identical number of amino acids as the corresponding wild type DNase I sequence SEQ ID NO: 1. In some aspects, a salt-tolerant DNase I may further comprise a heterologous amino acid sequence. In some aspects, a heterologous amino acid sequence may be covalently linked (i.e. fused) to a DNase I at an N-terminal or at a C-terminal end of a DNase I amino acid sequence. In some aspects, a heterologous amino acid sequence comprises a sequence-nonspecific double-stranded DNA binding domain. In some aspects, a sequence-non specific double-stranded DNA binding domain is selected from the group consisting of a DNA binding domain from a Maf proto-oncogene transcription factor, an Sso family DNA binding protein and a HMf transcription factor. Such domains are described for example in U.S. Pat. No. 8,535,925. In some aspects, a sequence-nonspecific double-stranded DNA binding domain comprises at least one helix-hairpin-helix motif. In some aspects, a heterologous amino acid sequence comprise a ComEA protein helix-hairpin-helix sequence. In some aspects, a ComEA protein helix-hairpin-helix sequence is from an organism of a genus selected from Bacillus, Thioalkalivibrio or Halomonas. ComEA proteins are described for example in U.S. Patent Public. No. 2017/0107501. In some aspects, a salt-tolerant DNase I comprising a heterologous amino acid sequence has DNase activity that is at least 10%, 20% 30%, 40%, 50% higher in a buffer comprising 600 mM, 800 mM or 1 M NaCl, as compared to a salt-tolerant DNase I without a heterologous amino acid sequence. In some aspects, a salt-tolerant DNase I comprising a heterologous amino acid sequence comprises a SEQ ID NO: 42.

### Thermolabile and Salt-Tolerant DNase

In some aspects, a mutant DNase I is thermolabile and salt-tolerant. In some aspects, a mutant DNase I comprises at least one substitution as compared to SEQ ID NO:1 in one or more amino acid positions selected from 5174 and T177. In some aspects, a mutant DNase I comprises one or more substitutions selected from S174K, S174R, T177R, T177K. In some aspects, a mutant DNase I comprises one or more substitutions selected from S174K and T177R. In some aspects, the activity of a mutant DNase I is about 20% or less after incubation for 20 min at 70° C. in a reaction buffer with 0 mM NaCl concentration, as compared to the activity prior to incubation. In some aspects, a mutant DNase I has from at least about 60% activity in a buffer comprising 100 mM NaCl as compared to the activity in a buffer comprising 0 mM NaCl concentration in the reaction buffer.

In some aspects, a mutant DNase I comprises a combination of substitutions selected from:

a) H44R, S75K, G105R, I130L, S138K, S174R, P197S, P227S,

b) H44R, S75K, G105R, I130L, S138K, S174K, P197S, T205R, P227S,

c) H44R, S75K, G105R, I130L, S138K, S174K, P197S, P227S,

d) T14K, S75K, G105R, I130L, S138K, S174K, P197S, T205R, or

e) T14K, S75K, G105R, I130L, S138K, S174K, P197S, P227S.

In some aspects, a mutant DNase I comprises SEQ ID NO: 37, SEQ ID NO: 38, SEQ ID NO: 39, SEQ ID NO: 40 or SEQ ID NO: 41.

In some aspects, the activity of a mutant DNase I is about 20% or less after incubation for 30 min at 60° C. in a reaction buffer with 0 mM NaCl concentration, as compared to the activity prior to incubation. In some aspects, a mutant DNase I has from at least about 100% to about 140% activity in a buffer comprising 100 mM NaCl as compared to the activity in a buffer comprising 0 mM NaCl concentration in the reaction buffer. That is, the mutant DNase I may be more active in the presence of 100 mM NaCl concentration in the reaction buffer than at 0 mM NaCl concentration.

In some aspects, a mutant DNase I comprises an amino acid sequence selected from SEQ ID NOs: 3, 6, 9, 12, 15, 17, 20, 23, 26, 28, 31.

### Kits and Compositions

In some aspects, a composition comprises a mutant DNase I according to the present disclosure and a suitable buffer. In some aspects, a composition comprises a mutant DNase I according to the present disclosure and a storage solution. In some aspects, a composition comprises a mutant DNase I according to the present disclosure and a buffer comprising at least one of Tris-HCl, CaCl₂, MgCl₂ and glycerol. In some aspects, a composition comprises a mutant DNase I according to the present disclosure and a buffer comprising at least one of Tris-HCl, CaCl₂, MgCl₂ and glycerol. In some aspects, a composition comprises a mutant DNase I according to the present disclosure and a buffer comprising at least Tris-HCl, CaCl₂, and glycerol.

In some aspects, a kit comprises a mutant DNase I according to present disclosure and a reaction buffer. In some aspects, a reaction buffer comprises at least 50 mM or at least 100 mM NaCl.

### Methods of Use

In a further aspect the present disclosure provides the use of a deoxyribonuclease according to the present disclosure to digest DNA in a sample. Similarly, a method is also provided for removing DNA from a sample comprising contacting the sample with the deoxyribonuclease of the present disclosure under conditions that allow the deoxyribonuclease to digest the DNA. In particular, the sample may comprise RNA.

A mutant DNase I of the present disclosure has particular utility in the following: (i) preparation of DNA-free RNA, (ii) removal of template DNA following in vitro transcription, (iii) preparation of DNA-free RNA prior to RT-PCR and RT-qPCR, (iv) DNA-free reagent preparation (also known as decontamination or sanitization of reagents), (v) DNA labeling by nick-translation in conjunction with DNA Polymerase I, (vi) studies of DNA-protein interactions by DNase I, RNase-free footprinting, or (vii) generation of a library of randomly overlapping DNA inserts.

As indicated above, a salt-tolerant DNase of the present disclosure may have higher activity compared to a bovine DNase having SEQ ID NO: 1 at NaCl concentrations of 50 mM, 100 mM and more, under otherwise equal conditions. In some aspects, a salt-tolerant DNase may have higher activity at 100 mM NaCl concentration as compared to the activity of salt-tolerant DNase at 0 mM NaCl concentration in the reaction buffer. As such, a salt-tolerant DNase I of the present disclosure may be efficiently utilized at higher salt concentrations than those utilized with the wild-type DNase I. Accordingly, in some aspects, in the method of the present invention the conditions that allow a DNase I to digest the DNA may include a concentration of from 50 mM to 600 mM NaCl. In some aspects, the concentration may be from 50 mM to 400 mM NaCl; in further aspects, the concentration may be 100 mM, 250 mM, 300 mM, 350 mM, 400 mM NaCl. In some aspects, in the method of the present invention the conditions that allow a DNase I to digest the DNA may include a concentration of from 50 mM to 600 mM of a salt other than NaCl. In some aspects, the concentration is from 50 mM to 400 mM NaCl; in further aspects, the concentration is 100 mM, 250 mM, 300 mM, 350 mM, 400 mM NaCl. In some aspects, a salt may be selected from sodium, potassium, lithium and ammonium salts. In some aspects, a salt may be selected from KCl, LiCl, (NH4)2SO4, Na2SO4. Various methods where a DNase I can be used may comprise concentration ranges of salt as listed above. For example, in vitro transcription or RT-PCR mixtures may comprise various salt concentrations, including the listed concentration ranges of salt. As another example, amplification reaction reagents for PCR or WGA, such as buffers, reaction mixtures, may comprise various salt concentrations, including the listed concentration ranges of salt.

In some aspects, a method for removing DNA from a sample comprising contacting the sample with the DNase I of the invention present disclosure under conditions that allow the deoxyribonuclease to digest the DNA, may further comprise a DNase I inactivation step. In some aspects, a DNase I inactivated after incubation at a temperature of 60° C., 65° C., and/or 70° C. for at least about 10 minutes, at least about 20 minutes or for more than 20 minutes (e.g. 30, 40, 50 or 60 minutes). In some aspects, a DNase I inactivated after incubation at a temperature of 60° C., 65° C., and/or 70° C. for 10 or 20 minutes. In some aspects, no EDTA is added prior to the incubation at a temperature of 60° C., 65° C., and/or 70° C.

It will be understood by one of ordinary skill level in the art that the amino acid sequences of mutant DNases I of the present invention are provided by way of example only and are not meant to limit the scope of the invention solely to those sequences explicitly spelled. Moreover, it is well within the purview of the skilled artisan to determine whether a given DNase I falls within the scope of the invention using standard techniques in the art, without requiring undue experimentation. Moreover, it will be appreciated that various variants of the DNase I sequences also fall within the scope of the invention, as long as such variants satisfy the structural and functional characteristics set forth above. Variants of DNases I not explicitly recited but satisfying the structural and functional requirements set forth above can include deletions, insertion or substitutions.

It will be readily apparent to one of ordinary skill in the relevant arts that other suitable modifications and adaptations to the methods and applications described herein are obvious and can be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1: Cloning and Purification of DNaseI Mutants

Bovine DNaseI was cloned using the aLICator kit (#K1251, Thermo Fisher Scientific). The single point mutations were constructed using a modified Stratagene QuikChange Site-Directed Mutagenesis (Agilent) approach. The constructs containing multiple mutations were constructed following the guidelines published in Hames C, et al. Appl Environ Microbiol. 2005 July; 71(7):4097-100. The constructs were cloned using E. coli ER2267 strain (New England Biolabs). For protein expression the constructed recombinant plasmids were transformed into E. coli ER2566 strain (New England Biolabs). The biomass was grown in 48 well plates using the EnPresso B kit (Merck). Bacteria were lysed chemically and the his-tagged proteins were purified in one step using nickel affinity purification with HisPur™ Ni-NTA Spin Plates (#88230, Thermo Fisher Scientific,). The washing buffer had the following composition: 2% Triton X-100; 20 mM Tris-HCl, pH 7.5; 0.5 M NaCl; 5 mM CaCl₂; 20 mM imidazole, pH 7.8. The elution buffer had the following composition: 2% Triton X-100; 20 mM Tris-HCl, pH 7.5; 0.5 M NaCl; 5 mM CaCl₂; 250 mM imidazole, pH 7.8. The enzymes were further transferred to the storage buffer (50% glycerol, 50 mM Tris-HCl pH 7.5, 10 mM CaCl₂, 0.1% Triton X-100) using gel filtration by Zeba™ Spin Desalting Plates (#89807, Thermo Fisher Scientific,). Protein purity was checked on SDS-PAGE (usually ˜40-80% of target protein), concentration was determined by densitometric analysis comparing to BSA standards.

### Example 2: Salt Tolerance and Thermolability Assays of Single Mutants

Thermolability and salt tolerance of DNaseI mutants was evaluated by analyzing digestion of fluorescently labeled DNA duplex (30 bp). As a substrate a fluorescent DNA duplex was used that was produced by hybridizing a quencher BHQ1-labeled oligonucleotide (SEQ ID NO: 43) and a reporter FAM-labeled oligonucleotide (SEQ ID NO: 44).

For thermolability assays, the diluted preparations of enzymes (dilution buffer: 0.5% Triton X-100, 50 mM Tris-HCl pH 7.5, 0.03% Elugent™, 0.2 mg/ml BSA) were split into several parts where one of those was kept at ˜4° C. and others were heated using varying temperatures and incubation times and then cooled back to ˜4° C. After such pretreatment the enzyme preparations were added to the reaction mix which resulted in the following composition: 10 mM Tris-HCl pH 7.5, 1 mM CaCl₂, 0.1% Triton X-100, 0.03% Elugent™, 0.2 mg/ml BSA, 0.025 μg/μl NoLimits 35 bp DNA Fragment (#5M1411, Thermo Fisher Scientific), 0.02 μM of the fluorescent DNA duplex, 5.85 ng/ml of the enzyme. Reactions were started by addition of 5× start solution containing 50 mM MgSO₄. The fluorescence was monitored at equal intervals for 10 minutes. Data (subsets of consecutive 2-4 data points) was fitted into first order equation and a maximum fluorescence change rate was calculated (Gen5™ Reader Control and Data Analysis Software), which was proportional to enzymatic activity. Fluorescence was monitored and start solution was distributed across reaction mixes by Synergy 2 Multi-Mode Reader (BioTek). Reaction mixes were prepared and fluorescence measures were taken at 23° C.

For salt tolerance assays, the experiments were performed by varying amounts of NaCl in the same reaction buffer in order to estimate activity changes due to the presence of varying amounts of salt in the reaction buffer. The reactions were started and the fluorescence of the solution was monitored in the same manner as in thermolability assays.

The summarized results of mutant DNases I activity at various reaction and inactivation conditions are provided in Table 1.

TABLE 1

| | | | | | | Salt tolerance | | |
|---|---|---|---|---|---|---|---|---|
| | | | | | | | 8 | |
| | | 3 | Thermolability | | | | Activity | |
| | | Activity | 4 | 5 | 6 | 7 | change | 9 |
| | 2 | relative | Activity | Activity | Activity | Activity | 100 mM | Remaining |
| | Activity | to his- | change | change | change | at 100 | vs 0 | activity at |
| 1 | relative to | wtDNase | 70° C. | 70° C. | 65° C. | mM | mM | 100 mM |
| Substitution | wtDNase I | I | 20′ | 10′ | 20′ | NaCl | NaCl | NaCl |
| wtDNase I | 100% | 145% | −1% | 5% | 15% | 40% | −72% | 28% |
| his-wtDNase I | 60.6% | 100% | −70% | −45% | 10% | 30% | −70% | 30% |
| T 14 K | 97.0% | 160% | −55% | −35% | −7% | 115% | −28% | 72% |
| T 14 R | 121.2% | 200% | −75% | −50% | −10% | 100% | −50% | 50% |
| H 44 R | 115.2% | 190% | −60% | −35% | 0% | 125% | −34% | 66% |
| S 75 K | 84.8% | 140% | −75% | −45% | −5% | 70% | −50% | 50% |
| G 105 R | 106.1% | 175% | −100% | −95% | −15% | 40% | −77% | 23% |
| I 130 L | 106.1% | 175% | −100% | −90% | −20% | 45% | −74% | 26% |
| S 138 K | 103.0% | 170% | −75% | −50% | −5% | 90% | −47% | 53% |
| S 174 K | 145.5% | 240% | −85% | −70% | −5% | 145% | −40% | 60% |
| T 177 R | 100.0% | 165% | −85% | −75% | −10% | 120% | −27% | 73% |
| P 197 S | 90.9% | 150% | −95% | −80% | −10% | 40% | −73% | 27% |
| T 205 R | 75.8% | 125% | −100% | −100% | −10% | 30% | −76% | 24% |
| P 227 S | 109.1% | 180% | −100% | −95% | −15% | 65% | −64% | 36% |

Columns 2 and 3 provide DNase activity as measured under reaction conditions without pretreatment with higher temperature and in the presence of 0 mM NaCl. Column 2 shows the activity of tested DNases in comparison to wtDNase I (SEQ ID NO: 1), the reference activity of which is indicated as 100%. For example, a DNase I mutant that has an activity of 115% is 1.15 times more active than wtDNase I, under the same conditions. Column 3 of the Table 1 denotes the activity of tested DNases in comparison to his-wtDNase I (SEQ ID NO: 2), the reference activity of which is indicated as 100%. For example, a DNase I mutant that has an activity of 160% is 1.6 times more active than his-wtDNase I, under the same conditions.

As can be seen from the results in Table 1, DNase I with a single substitution at a position 105, 130, 174, 177, 197, 205, or 227 had increased thermolability and were either inactive (activity change −100%) or were inactivated to a larger extent compared to the inactivation of wild type DNases after incubation under the same conditions (at 70° C. for 20 minutes (column 4) or 70° C. 10 minutes (column 5)). The change in activity upon heating as indicated in columns 4, 5 and 6 was calculated according to Formula I:

[[(Activity (heated)−Activity (not-heated)]/Activity (not-heated)]×100%

Accordingly, DNase I with a single substitution at a position 14, 44, 75, 138, 174, or 177 showed increased salt tolerance compared to the wild type DNases after reaction under the same conditions (in the presence of 100 mM NaCl). This can be seen from the columns 7 and 8 of Table 1. Some The change of activity at 100 mM NaCl concentration (column 8) was calculated according to the Formula II:

[[(Activity (100 mM)−Activity (0 mM)]/Activity (0 mM)]×100%

The DNase I mutants with a substitution at position 174 or 177 showed both improved properties—increased thermolability and increased salt tolerance.

### Example 3: Construction and Analysis of Multiple Mutant DNase I Variants

DNase I multiple mutants with various combinations of the mutations providing thermolability or salt tolerance, respectively, were constructed, expressed and purified as described above, and their activity (thermolability and/or salt tolerance) was tested under the same conditions as above. The activity of thermolabile mutant DNases Mut16 (I130L; P197S; T205R) and Mut18 (G105R; I130L; P197S; P227S) decreased by about 99% after incubation at 65° C. for 20 minutes, as calculated using Formula I (data not shown). This is substantial increase in thermolability, when compared to the wild type DNase I, the activity of which decreased only by 10 to 15% under the same conditions.

The salt-tolerant mutant DNases Mut4 (T14K; S75K; G105R; S138K; S174K; P197S) and Mut13 (H44R; S75K; G105R; S138K; S174K; P197S; P227) showed about 2 times (i.e. about 100% activity as calculated according to Formula II) increased activity under reaction conditions with 100 mM NaCl compared to 0 mM NaCl (data not shown). Thus, these mutant DNases not only did not lose their activity in the presence of 100 mM NaCl, but they were more active in comparison to the reaction conditions with 0 mM NaCl.

Further, the multiple mutant DNase I variants that would potentially have both thermolability and salt tolerance increased, were constructed. The activity of the mutant DNases was determined using the same methods and activity change calculation formulas as in previous examples. As can be seen from the results provided in Table2, mutant DNases #III and #V show the most increased thermolability, as their activity drops by more than 90% after incubation for 20 minutes at 65° C., and more than 50% after incubation for 20 minutes at 55° C. At the same time #III and #V have increased salt tolerance—their activity rises in the presence of 100 mM NaCl, as compared to 0 mM NaCl.

TABLE 2

| | | | | Salt tolerance |
|---|---|---|---|---|
| | | Thermolability | | Activity |
| | | Activity | Activity | change** |
| | Mutant DNase I | change* | change* | 100 mM vs 0 |
| # | Substitutions | 65° C. 20′ | 55° C. 20′ | mM NaCl |
| I | T14K; S75K; G105R; | −9% | 0% | 170% |
| | I130L; S138K; S174K; | | | |
| | P197S; T205R | | | |
| II | T14K; S75K; G105R; | −30% | 0% | 100% |
| | I130L; S138K; S174K; | | | |
| | P197S; P227S | | | |
| III | H44R; S75K; G105R; | −92% | −55% | 130% |
| | I130L; S138K; S174K; | | | |
| | P197S; T205R; P227S | | | |
| IV | H44R; S75K; G105R; | −88% | 0% | 80% |
| | I130L; S138K; S174K; | | | |
| | P197S; P227S | | | |
| V | H44R; S75K; G105R; | −98% | −60% | 30% |
| | I130L; S138K; S174R; | | | |
| | P197S; P227S | | | |

*calculated according to Formula I;
**calculated according to Formula II;

### Example 4: Purification of Multiple DNase Mutants

To further investigate the properties of mutant variants of DNase I, mutants #III (SEQ ID NO: 39), #V (SEQ ID NO: 41) and a DNase I mutant #V fused to ComEA domain from Bacillus stearothermophilus (the fused DNase I #V-ComEA sequence is SEQ ID NO: 42), as well as the wild type DNase I (SEQ ID NO: 1) were expressed in yeast for higher yield. For this, each DNase I was expressed in PichiaPink™ expression system (Thermo Fisher Scientific; pPink-HC vector, serum albumin signal sequence, PichiaPink™ Strain 1) and it was actively secreted into the culture medium. Protein expression was conducted in a Biostat A (Sartorius) bioreactor where BMGY (Buffered Glycerol-complex Medium) culture medium was inoculated with overnight culture of PichiaPink cells. During fed-batch phase with glycerol the temperature was kept at 30° C., pH 6, DO (Dissolved oxygen) 30% and stirring was 200 rpm. Culture feeding with glycerol was stopped after 12 hours, temperature was reduced to 28° C. and methanol feed was initiated and continued for another 24-48 hours. PichiaPink cells were removed by performing microfiltration and culture supernatant was concentrated and buffer exchanged into 20 mM sodium acetate, 1 mM calcium chloride and 250 mM sodium chloride at pH 5 by using tangential flow filtration (TFF). Subsequently, DNase I was purified by using Poros XS and Poros Benzyl resins (Thermo Fisher Scientific) on Akta Pure chromatography system (GE Healthcare Life Sciences), resulting in more than 95% purity. DNase I mutant activity units were determined by diluting DNase 20-100 times and incubating in a reaction buffer: 20 mM Tris-HCl pH 7.6, 0.1 mM CaCl₂, 100 mM NaCl (0 mM NaCl in case of wild type DNase I), 0.1% Elugent™, 15 mM MgCl₂, 0.5 μg/μl BSA, 0.025 μg/μl NoLimits 35 bp DNA Fragment (#5M1411, Thermo Fisher Scientific), 0.02 μM Fluorescent DNA duplex (SEQ ID NO: 43 annealed to SEQ ID NO: 44). Reaction was performed at 23° C. for 90 minutes, and fluorescence values were periodically detected. The reaction rate (RFU/s) is calculated, compared to the reaction rate of reference protein Turbo™ DNase (AM2238, Thermo Fisher Scientific) and the activity units U/μl are calculated according to the reference protein.

### Example 5: Salt Tolerance Studies of Multiple DNase Mutants

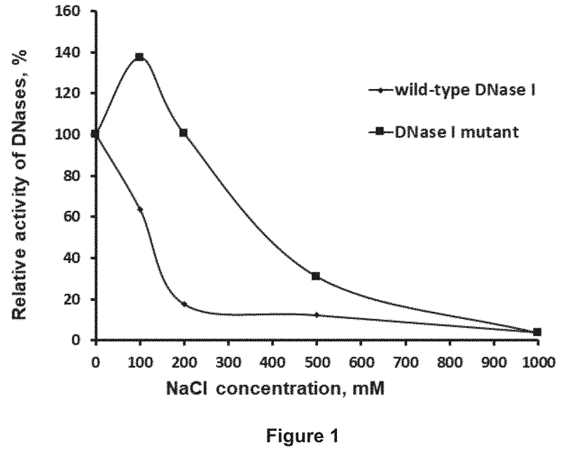

The reaction was conducted at 37° C. for 30 min in the presence of 1 mg/ml herring sperm DNA and in the presence of different concentrations of sodium chloride (0-1M of NaCl). Reaction conditions: 40 mM Tris-HCl pH 8.0, 10 mM MgCl₂, 1 mM CaCl₂. 1 U/μ1 of each DNase-wild type DNase I (SEQ ID NO: 1) and DNase I mutant #V (SEQ ID NO: 41) was diluted 250× in a final reaction mixture of 682.5 The reaction was stopped by addition of 4% perchloric acid in a ratio 1:1, kept on ice for 30 min and centrifuged for 6 min at 14,000×g. Afterwards the absorbance at 260 nm was measured and the values of remaining undigested DNA were recalculated to represent the residual activity of DNase. The results are presented in FIG. 1; as can be seen from the figure, the DNase I mutant retains its activity at 100 mM NaCl concentration and is about 40% more active under such conditions compared to 0 mM NaCl, and the activity starts to drop only at concentrations higher than 200 mM NaCl. In comparison, the wild type DNase I loses about 40% of its activity under at 100 mM NaCl concentration under the tested conditions.

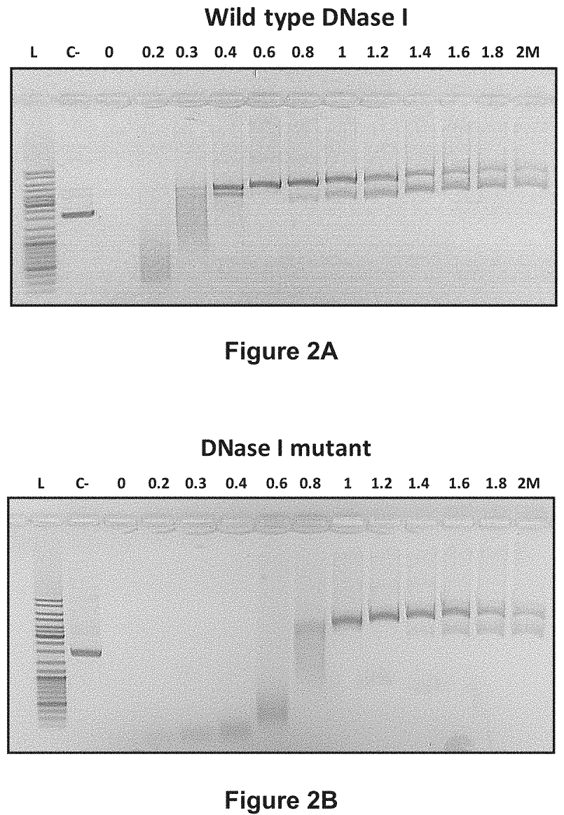

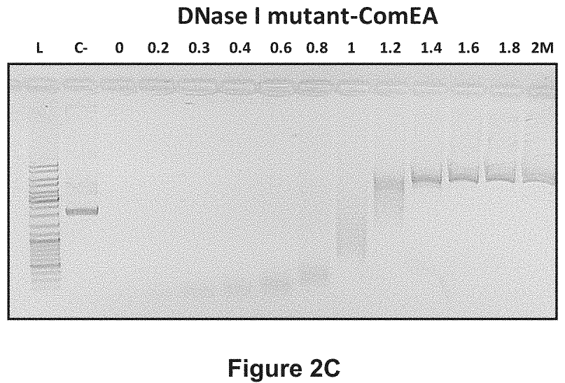

Next, 1 μg of plasmid pUC19 DNA was used as another substrate for DNase I. The reaction was conducted under the same conditions as with herring sperm DNA. In addition to wild type DNase I and DNase I mutant #V, a DNase I mutant #V-ComEA (SEQ ID NO: 42) was assayed. The reaction was stopped by adding 6×DNA Loading Dye and SDS Solution (R1151, Thermo Fisher Scientific) and inactivated by heating at 70° C. for 5 min. Samples were run on 1% agarose gels. The results are presented in FIG. 2; as can be seen from the figure, the DNase I mutant #V can digest plasmid DNA at NaCl concentrations up to 1 M NaCl under the presented conditions (a band seen in the 1 M NaCl lane represents a nicked form of the plasmid), whereas the wild type DNase I lost majority of its activity and was able to digest the plasmid DNA to some extent only at lower NaCl concentrations of 0.6-0.8 M (FIG. 2A). Also, as can be seen from FIG. 2C, ComEA domain when fused to DNase I, contributes to further increased salt-tolerance of the enzyme, as compared to the non-fused DNase I in FIG. 2B.

### Example 6: Thermolability Experiments

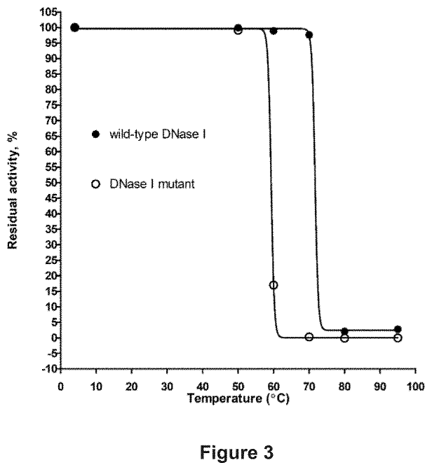

Wild type DNase and DNase I mutant #V were incubated for 30 min at different temperatures (4° C., 50° C., 60° C., 70° C., 80° C., 95° C.) in a buffer (50 mM Tris-HCl pH 8, 0.1% Triton X-100, 1 mM CaCl₂) before conducting the DNase assay. The DNase assay was conducted at 37° C. for 30 min in the presence of 1 mg/ml herring sperm DNA. Reaction conditions: 40 mM Tris-HCl pH 8.0, 10 mM MgCl₂, 1 mM CaCl₂. 1U/μl of each DNase was diluted 250× in a final reaction mixture. The reaction was stopped by addition of 4% perchloric acid in a ratio 1:1, kept on ice for 30 min and centrifuged for 6 min at 14,000×g. Afterwards the absorbance at 260 nm was measured and the values of remaining undigested DNA were recalculated to represent the residual activity of DNase. The results are presented in FIG. 3; as can be seen from the figure, the DNase I mutant #V loses more than 80% of its activity after pre-incubation at 60° C. for 30 minutes, and is fully inactivated after pre-incubation at 70° C. for 30 minutes. In comparison, the wild type DNase I retains about 100% activity after pre-incubation at 60° C. for 30 minutes, and has more than 95% of its activity after pre-incubation at 70° C. for 30 minutes.

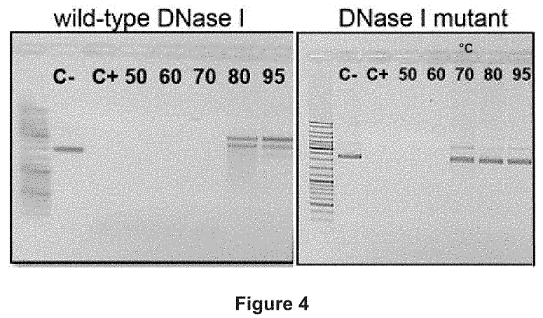

1 μg of plasmid pUC19 DNA was used as another substrate for DNase I. The reaction was conducted under the same conditions as with herring sperm DNA. The reaction was stopped by adding 6×DNA Loading Dye and SDS Solution (R1151, Thermo Fisher Scientific) and inactivated by heating at 70° C. for 5 min. Samples were run on 1% agarose gels. The results are presented in FIG. 4; as can be seen from the FIG. 4, after pre-incubation at 70° C. the DNase I mutant #V is inactivated and cannot digest plasmid DNA, whereas the wild type DNase I is not inactivated after pre-incubation at 70° C. and also retains some activity after pre-incubation at 80° C., as can be seen from the “smear” of DNA in the 80° C. lane in FIG. 4.

### Example 7: DNA Removal from In Vitro Transcription Reactions

In vitro transcription reactions were performed using TranscriptAID T7 High Yield Transcription kit (K0441, Thermo Fisher Scientific), according to the instruction manual; 1 μg of Control template DNA provided with the kit was used. After IVT reactions 1 U of wild type DNase I or DNase I mutant #V was added and reaction was incubated for 15 min at 37° C., then EDTA was added to 15 mM and DNases were heat inactivated by incubating them at 75° C. for 10 min. Residual template DNA was measured by using Maxima SYBR Green qPCR Master Mix (Thermo Fisher Scientific) and a pair of specific primers (SEQ ID NO: 45 and SEQ ID NO: 46) targeting DNA template. DNase I mutant #V efficiently removed the template DNA (the residual DNA was calculated to be about 1% of initial DNA amount), while the same amounts of wild type DNase I were not sufficient to digest the template DNA, leaving more than 95% of the template DNA undigested. While the TranscriptAID T7 High Yield Transcription kit provides instructions to inactivate the DNase I (if used) by phenol/chloroform extraction, using thermolabile DNase I mutant #V allowed the thermal inactivation, thus avoiding laborious extraction step.

### Example 8: Sanitization of Reaction Components

DNase I mutant #III was used in sanitization reaction, during which the DNA decontamination of reagents for whole genome amplification (WGA) reaction was performed. DNA contamination is especially important in applications where very small amounts of DNA (e.g. single cell analysis) are amplified for further analysis. As little as a single copy or a few copies of contaminating DNA originating from reagents, plastics or reaction setup can be amplified leading to false-positive results.

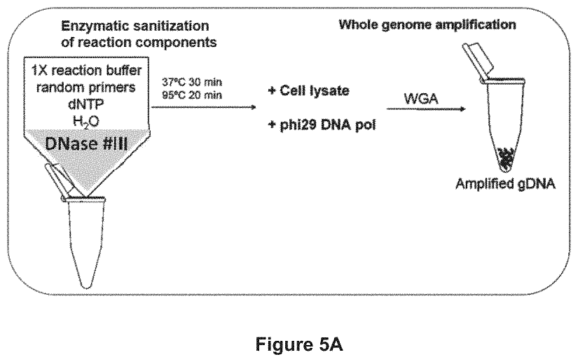

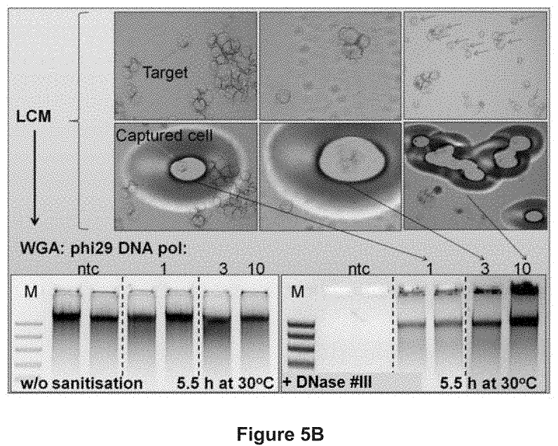

A solution containing 1× phi29 DNA Polymerase reaction buffer (10× buffer composition: 330 mM Tris-acetate (pH 7.9 at 37° C.), 100 mM Mg-acetate, 660 mM K-acetate, 1% (v/v) Tween 20, 10 mM DTT; #EP0091, Thermo Fisher Scientific), 1 mM dNTP, 25 μM Exo-Resistant Random Primer (#S0181, Thermo Fisher Scientific) and 0.2 U/μl of DNase I #III was incubated for 30 minutes at 37° C. After incubation, the reaction was inactivated for 20 minutes at 90° C. 1, 3 and 10 HeLa cells were taken using Laser capture microdissection (LCM), and cells were lysed using Single Cell Lysis Solution from Single Cell Lysis Kit (Thermo Fisher Scientific). Each of the lysates were combined with the DNase I-treated solution and 0.5 U/μl phi29 DNA Polymerase (#EP0091, Thermo Fisher Scientific), and resulting reaction mixtures were incubated for 5.5 hours at 30° C. In parallel, the reactions at equal conditions and of equal composition were incubated, except that the reaction solution was not pre-treated with a DNase I. No-template reactions (NTC) which did not contain cell lysates were performed in both cases. The reaction results were analysed in electrophoresis gel and are provided in FIG. 5B. As can be seen in the electrophoresis gel pictures in the bottom of FIG. 5B, the NTC reaction, the reaction solution of which was not pre-treated with DNase I #III, provided amplification product. This means that the reaction mixture contained traces of DNA which was amplified by phi29 DNA polymerase, and therefore, at least part of amplification product that can be seen after in case of amplification of template DNA from cell lysates may be false-positive results. In case, where the reaction solution was pre-treated with DNase I #III, the NTC gel lanes do not show any amplification, indicating that any traces of DNA that could be contained in the reaction solution prior to addition of template DNA from cell lysates, were digested by DNase I #III.

All publications, patents and patent applications mentioned in this specification are herein incorporated in their entirety by reference into the specification, to the same extent as if each individual publication, patent or patent application was specifically and individually indicated to be incorporated herein by reference. In case of conflict, the specification herein, including definitions, will control. Citation or identification of any reference in this application shall not be construed as an admission that such reference is available as prior art to the present invention.

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A deoxyribonuclease comprising one or more substitutions at the following selected amino acid positions T14, H44, S75, G105, I130, S138, S174, T177, P197, T205, P227 corresponding to SEQ ID NO: 1, and having at least 80%, 85%, 90%, 95%, 98% or 99% sequence identity with SEQ ID NO: 1.

2. The deoxyribonuclease of claim 1, having at least 25% of the activity of a deoxyribonuclease having SEQ ID NO: 1 at 23° C. in a buffer comprising 0 mM NaCl.

3. The deoxyribonuclease of claim 1, wherein the deoxyribonuclease maintains 30% or less activity after incubation at 70° C. for 20 minutes.

4. The deoxyribonuclease of any of claims 1 to 3, wherein the deoxyribonuclease maintains at least 30% activity in a buffer comprising 100 mM NaCl.

5. The deoxyribonuclease of any previous claim, wherein the one or more substitutions are selected from T14K, T14R, H44R, H44K, S75K, S75R, G105R, G105K, I130L, I130V, I130M, S138K, S138R, S174K, S174R, T177R, T177K, P197S, T205R, T205K, P227S.

6. The deoxyribonuclease of any previous claim, wherein the one or more substitutions are selected from G105, I130, S174, T177, P197, T205, P227.

7. The deoxyribonuclease of any previous claim, wherein the one or more substitutions are selected from G105R, G105K, I130L, I130V, I130M, S174K, S174R, T177R, T177K, P197S, T205R, T205K, P227S.

8. The deoxyribonuclease of any previous claim, wherein the one or more substitutions are selected from G105R, I130L, S174K, S174R, T177R, P197S, T205R, P227S.

9. The deoxyribonuclease of any previous claim, wherein the one or more substitutions are selected from G105R, I130L, P197S, T205R, P227S.

10. The deoxyribonuclease of claim 9, comprising the substitutions I130L, P197S and T205R.

11. The deoxyribonuclease of claim 9, comprising the substitutions G105R, I130L, P197S, T205R and P227S.

12. The deoxyribonuclease of any of claims 1 to 5, wherein the one or more substitutions are selected from T14, H44, S75, S138, S174, T177.

13. The deoxyribonuclease of claim 12, wherein the one or more substitutions are selected from T14K, T14R, H44R, H44K, S75K, S75R, S138K, S138R, S174K, S174R, T177R, T177K.

14. The deoxyribonuclease of claim 13, comprising the substitutions S75K, S138K, S174K.

15. The deoxyribonuclease of claim 14, further comprising one or more substitutions selected from T14K, H44R, G105R, P197S, P227S.

16. The deoxyribonuclease of any of claims 1 to 4, comprising a combination of substitutions selected from:
a) H44R, S75K, G105R, I130L, S138K, S174R, P197S, P227S,
b) H44R, S75K, G105R, I130L, S138K, S174K, P197S, T205R, P227S,
c) H44R, S75K, G105R, I130L, S138K, S174K, P197S, P227S,
d) T14K, S75K, G105R, I130L, S138K, S174K, P197S, T205R, or
e) T14K, S75K, G105R, I130L, S138K, S174K, P197S, P227S.

17. A deoxyribonuclease having an amino acid sequence comprising SEQ ID NO: 37, SEQ ID NO: 38, SEQ ID NO: 39, SEQ ID NO: 40 or SEQ ID NO: 41.

18. The deoxyribonuclease of any previous claim, wherein the deoxyribonuclease further comprises a heterologous amino acid sequence.

19. The deoxyribonuclease of claim 18, wherein heterologous amino acid sequence comprises a sequence-nonspecific double-stranded DNA binding domain.

20. The deoxyribonuclease of claim 19, wherein the DNA binding domain comprises at least one helix-hairpin-helix motif.

21. The deoxyribonuclease of claim 20, wherein the heterologous amino acid sequence comprises a ComEA protein helix-hairpin-helix sequence.

22. The deoxyribonuclease of claim 21, wherein the ComEA protein helix-hairpin-helix sequence is from an organism of a genus selected from Bacillus, Thioalkalivibrio or Halomonas.

23. A composition comprising a deoxyribonuclease of any previous claim and a buffer.

24. A composition according to claim 23, wherein the buffer comprises at least one of Tris-HCl, CaCl₂, MgCl₂ and glycerol.

25. A kit for removing DNA from a sample comprising a deoxyribonuclease according to any of claims 1 to 22 and a reaction buffer.

26. A kit of claim 25, wherein a reaction buffer comprises at least 50 mM or at least 100 mM NaCl.

27. Use of a deoxyribonuclease according to any one of claims 1 to 22 or a kit according to any one of claim 25 or 26 to digest DNA in a sample.

28. Use according to claim 27, wherein the sample comprises RNA.

29. A method for removing DNA from a sample comprising contacting the sample with the deoxyribonuclease of any one of claims 1 to 22 under conditions that allow the deoxyribonuclease to digest the DNA.

30. A method according to claim 29, wherein the conditions include from 50 mM to 600 mM NaCl.

31. A method according to claim 29 or claim 30, wherein the sample comprises RNA.


SEQUENCE LISTING (47 sequences)

SEQ ID NO: 1 - protein, 260 aa, Bos sp.
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 2 - protein, 290 aa, Artificial Sequence
  source: /note=“Description of Artificial Sequence Synthetic polypeptide”
MAGSHHHHHHGMASMTGGQQMGRSGDDDDKLKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 3 - protein, 260 aa, Artificial Sequence
  source: /note=“Description of Artificial Sequence Synthetic polypeptide”
  VARIANT (14)..(14): /replace=“Arg” or “Gln” or “Asn” or “His”
  SITE (1)..(260): /note=“Variant residues given in the sequence have no preference with respect to those in the annotations for variant positions”
LKIAAFNIRTFGEKKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 4 - protein, 260 aa, Artificial Sequence; identical to SEQ ID NO: 3
  source: /note=“Description of Artificial Sequence Synthetic polypeptide”

SEQ ID NO: 5 - protein, 260 aa, Artificial Sequence
  source: /note=“Description of Artificial Sequence Synthetic polypeptide”
LKIAAFNIRTFGERKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 6 - protein, 260 aa, Artificial Sequence
  source: /note=“Description of Artificial Sequence Synthetic polypeptide”
  VARIANT (44)..(44): /replace=“Arg” or “Gln” or “Asn”
  SITE (1)..(260): /note=“Variant residues given in the sequence have no preference with respect to those in the annotations for variant positions”
LKIAAFNIRTFGETKMSNATLASYIVRIVRRYDIVLIQEVRDSKLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 7 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSRLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 8 - protein, 260 aa, Artificial Sequence; identical to SEQ ID NO: 6
  source: /note=“Description of Artificial Sequence Synthetic polypeptide”

SEQ ID NO: 9 - protein, 260 aa, Artificial Sequence
  source: /note=“Description of Artificial Sequence Synthetic polypeptide”
  VARIANT (75)..(75): /replace=“Arg” or “Gln” or “Asn” or “His”
  SITE (1)..(260): /note=“Variant residues given in the sequence have no preference with respect to those in the annotations for variant positions”
LKIAAFNIRTFGETKMSNATLASYIVRIVRRYDIVLIQEVRDSHLVAVGKLLDYLNQDDPNTYHYVVSEPLGRNKYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 10 - protein, 260 aa, Artificial Sequence; identical to SEQ ID NO: 9
  source: /note=“Description of Artificial Sequence Synthetic polypeptide”

SEQ ID NO: 11 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSHLVAVGKLLDYLNQDDPNTYHYVVSEPLGRNRYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 12 - protein, 260 aa, Artificial Sequence
  source: /note=“Description of Artificial Sequence Synthetic polypeptide”
  VARIANT (105)..(105): /replace=“Lys” or “Gln” or “Asn” or “His”
  SITE (1)..(260): /note=“Variant residues given in the sequence have no preference with respect to those in the annotations for variant positions”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCRNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 13 - protein, 260 aa, Artificial Sequence; identical to SEQ ID NO: 12
  source: /note=“Description of Artificial Sequence Synthetic polypeptide”

SEQ ID NO: 14 - protein, 260 aa, Artificial Sequence; identical to SEQ ID NO: 12
  source: /note=“Description of Artificial Sequence Synthetic polypeptide”

SEQ ID NO: 15 - protein, 260 aa, Artificial Sequence
  source: /note=“Description of Artificial Sequence Synthetic polypeptide”
  VARIANT (130)..(130): /replace=“Val” or “Leu”
  SITE (1)..(260): /note=“Variant residues given in the sequence have no preference with respect to those in the annotations for variant positions”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAMVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 16 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFALVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 17 - protein, 260 aa, Artificial Sequence
  source: /note=“Description of Artificial Sequence Synthetic polypeptide”
  VARIANT (138)..(138): /replace=“Arg” or “Gln” or “Asn” or “His”
  SITE (1)..(260): /note=“Variant residues given in the sequence have no preference with respect to those in the annotations for variant positions”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPKDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 18 - protein, 260 aa, Artificial Sequence; identical to SEQ ID NO: 17
  source: /note=“Description of Artificial Sequence Synthetic polypeptide”

SEQ ID NO: 19 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPRDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 20 - protein, 260 aa, Artificial Sequence
  source: /note=“Description of Artificial Sequence Synthetic polypeptide”
  VARIANT (174)..(174): /replace=“Arg” or “Gln” or “Asn” or “His”
  SITE (1)..(260): /note=“Variant residues given in the sequence have no preference with respect to those in the annotations for variant positions”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCKYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 21 - protein, 260 aa, Artificial Sequence; identical to SEQ ID NO: 20
  source: /note=“Description of Artificial Sequence Synthetic polypeptide”

SEQ ID NO: 22 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCRYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 23 - protein, 260 aa, Artificial Sequence
  source: /note=“Description of Artificial Sequence Synthetic polypeptide”
  VARIANT (177)..(177): /replace=“Lys” or “Gln” or “Asn” or “His”
  SITE (1)..(260): /note=“Variant residues given in the sequence have no preference with respect to those in the annotations for variant positions”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVRSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 24 - protein, 260 aa, Artificial Sequence; identical to SEQ ID NO: 23
  source: /note=“Description of Artificial Sequence Synthetic polypeptide”

SEQ ID NO: 25 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVKSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 26 - protein, 260 aa, Artificial Sequence
  source: /note=“Description of Artificial Sequence Synthetic polypeptide”
  VARIANT (197)..(197): /replace=“Arg” or “Asn” or “Asp” or “Cys” or “Glu” or “Gln” or “Gly” or “His” or “Ile” or “Leu” or “Lys” or “Met” or “Phe” or “Pro” or “Thr” or “Trp” or “Tyr” or “Val”
  SITE (1)..(260): /note=“Variant residues given in the sequence have no preference with respect to those in the annotations for variant positions”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLISDSADTTATSTNCAYDRIVVAGSLLQSSVVPGSAAPFDFQAAYGLSNEMALAISDHYPVEVTLT

SEQ ID NO: 27 - protein, 260 aa, Artificial Sequence; identical to SEQ ID NO: 26
  source: /note=“Description of Artificial Sequence Synthetic polypeptide”

SEQ ID NO: 28 - protein, 260 aa, Artificial Sequence
  source: /note=“Description of Artificial Sequence Synthetic polypeptide”
  VARIANT (205)..(205): /replace=“Lys” or “Gln” or “Asn” or “His”
  SITE (1)..(260): /note=“Variant residues given in the sequence have no preference with respect to those in the annotations for variant positions”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTARSTNCAYDRIVVAGSLLQSSVVPGSAAPFDFQAAYGLSNEMALAISDHYPVEVTLT

SEQ ID NO: 29 - protein, 260 aa, Artificial Sequence; identical to SEQ ID NO: 28
  source: /note=“Description of Artificial Sequence Synthetic polypeptide”

SEQ ID NO: 30 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTAKSTNCAYDRIVVAGSLLQSSVVPGSAAPFDFQAAYGLSNEMALAISDHYPVEVTLT

SEQ ID NO: 31 - protein, 260 aa, Artificial Sequence
  source: /note=“Description of Artificial Sequence Synthetic polypeptide”
  VARIANT (227)..(227): /replace=“Arg” or “Asn” or “Asp” or “Cys” or “Glu” or “Gln” or “Gly” or “His” or “Ile” or “Leu” or “Lys” or “Met” or “Phe” or “Pro” or “Thr” or “Trp” or “Tyr” or “Val”
  SITE (1)..(260): /note=“Variant residues given in the sequence have no preference with respect to those in the annotations for variant positions”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVSGSAAPFDFQAAYGLSNEMALAISDHYPVEVTLT

SEQ ID NO: 32 - protein, 260 aa, Artificial Sequence; identical to SEQ ID NO: 31
  source: /note=“Description of Artificial Sequence Synthetic polypeptide”

SEQ ID NO: 33 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFALVALHSAPSDAVAEINSLYDVYLDVQQKWHLNDVMLMGDFNADCSYVTSSQWSSIRLRTSSTFQWLISDSADTTARSTNCAYDRIVVAGSLLQSSVVPGSAAPFDFQAAYGLSNEMALAISDHYPVEVTLT

SEQ ID NO: 34 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCRNDSFSREPAVVKFSSHSTKVKEFALVALHSAPSDAVAEINSLYDVYLDVQQKWHLNDVMLMGDFNADCSYVTSSQWSSIRLRTSSTFQWLISDSADTTATSTNCAYDRIVVAGSLLQSSVVSGSAAPFDFQAAYGLSNEMALAISDHYPVEVTLT

SEQ ID NO: 35 - protein, 260 aa, Artificial Sequence
  source: /note=“Description of Artificial Sequence Synthetic polypeptide”
LKIAAFNIRTFGEKKMSNATLASYIVRIVRRYDIVLIQEVRDSHLVAVGKLLDYLNQDDPNTYHYVVSEPLGRNKYKERYLFLFRPNKVSVLDTYQYDDGCESCRNDSFSREPAVVKFSSHSTKVKEFAIVALHSAPKDAVAEINSLYDVYLDVQQKWHLNDVMLMGDFNADCKYVTSSQWSSIRLRTSSTFQWLISDSADTTATSTNCAYDRIVVAGSLLQSSVVPGSAAPFDFQAAYGLSNEMALAISDHYPVEVTLT

SEQ ID NO: 36 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSRLVAVGKLLDYLNQDDPNTYHYVVSEPLGRNKYKERYLFLFRPNKVSVLDTYQYDDGCESCRNDSFSREPAVVKFSSHSTKVKEFAIVALHSAPKDAVAEINSLYDVYLDVQQKWHLNDVMLMGDFNADCKYVTSSQWSSIRLRTSSTFQWLISDSADTTATSTNCAYDRIVVAGSLLQSSVVSGSAAPFDFQAAYGLSNEMALAISDHYPVEVTLT

SEQ ID NO: 37 - protein, 260 aa, Artificial Sequence
  source: /note=“Description of Artificial Sequence Synthetic polypeptide”
LKIAAFNIRTFGEKKMSNATLASYIVRIVRRYDIVLIQEVRDSHLVAVGKLLDYLNQDDPNTYHYVVSEPLGRNKYKERYLFLFRPNKVSVLDTYQYDDGCESCRNDSFSREPAVVKFSSHSTKVKEFALVALHSAPKDAVAEINSLYDVYLDVQQKWHLNDVMLMGDFNADCKYVTSSQWSSIRLRTSSTFQWLISDSADTTARSTNCAYDRIVVAGSLLQSSVVPGSAAPFDFQAAYGLSNEMALAISDHYPVEVTLT

SEQ ID NO: 38 - protein, 260 aa, Artificial Sequence
  source: /note=“Description of Artificial Sequence Synthetic polypeptide”
LKIAAFNIRTFGEKKMSNATLASYIVRIVRRYDIVLIQEVRDSHLVAVGKLLDYLNQDDPNTYHYVVSEPLGRNKYKERYLFLFRPNKVSVLDTYQYDDGCESCRNDSFSREPAVVKFSSHSTKVKEFALVALHSAPKDAVAEINSLYDVYLDVQQKWHLNDVMLMGDFNADCKYVTSSQWSSIRLRTSSTFQWLISDSADTTATSTNCAYDRIVVAGSLLQSSVVSGSAAPFDFQAAYGLSNEMALAISDHYPVEVTLT

SEQ ID NO: 39 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSRLVAVGKLLDYLNQDDPNTYHYVVSEPLGRNKYKERYLFLFRPNKVSVLDTYQYDDGCESCRNDSFSREPAVVKFSSHSTKVKEFALVALHSAPKDAVAEINSLYDVYLDVQQKWHLNDVMLMGDFNADCKYVTSSQWSSIRLRTSSTFQWLISDSADTTARSTNCAYDRIVVAGSLLQSSVVSGSAAPFDFQAAYGLSNEMALAISDHYPVEVTLT

SEQ ID NO: 40 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSRLVAVGKLLDYLNQDDPNTYHYVVSEPLGRNKYKERYLFLFRPNKVSVLDTYQYDDGCESCRNDSFSREPAVVKFSSHSTKVKEFALVALHSAPKDAVAEINSLYDVYLDVQQKWHLNDVMLMGDFNADCKYVTSSQWSSIRLRTSSTFQWLISDSADTTATSTNCAYDRIVVAGSLLQSSVVSGSAAPFDFQAAYGLSNEMALAISDHYPVEVTLT

SEQ ID NO: 41 - protein, 260 aa, Artificial Sequence
  source: /note=“Description of Artificial Sequence Synthetic polypeptide”
LKIAAFNIRTFGETKMSNATLASYIVRIVRRYDIVLIQEVRDSRLVAVGKLLDYLNQDDPNTYHYVVSEPLGRNKYKERYLFLFRPNKVSVLDTYQYDDGCESCRNDSFSREPAVVKFSSHSTKVKEFALVALHSAPKDAVAEINSLYDVYLDVQQKWHLNDVMLMGDFNADCRYVTSSQWSSIRLRTSSTFQWLISDSADTTATSTNCAYDRIVVAGSLLQSSVVSGSAAPFDFQAAYGLSNEMALAISDHYPVEVTLT

SEQ ID NO: 42 - protein, 345 aa, Artificial Sequence
  source: /note=“Description of Artificial Sequence Synthetic polypeptide”
LKIAAFNIRTFGETKMSNATLASYIVRIVRRYDIVLIQEVRDSRLVAVGKLLDYLNQDDPNTYHYVVSEPLGRNKYKERYLFLFRPNKVSVLDTYQYDDGCESCRNDSFSREPAVVKFSSHSTKVKEFALVALHSAPKDAVAEINSLYDVYLDVQQKWHLNDVMLMGDFNADCRYVTSSQWSSIRLRTSSTFQWLISDSADTTATSTNCAYDRIVVAGSLLQSSVVSGSAAPFDFQAAYGLSNEMALAISDHYPVEVTLTGEETAVQQGGGGSVQSDGGKGALVNINTATLEELQGISGVGPSKAEAIIAYREENGRFQTIEDITKVSGIGEKSFEKIKSSITVK

SEQ ID NO: 43 - DNA, 30 bp, Artificial Sequence
  source: /note=“Description of Artificial Sequence Synthetic oligonucleotide”
gttggtgggtttgggtgtgggtttgtgttt

SEQ ID NO: 44 - DNA, 30 bp, Artificial Sequence
  source: /note=“Description of Artificial Sequence Synthetic oligonucleotide”
aaacacaaacccacacccaaacccaccaac

SEQ ID NO: 45 - DNA, 22 bp, Artificial Sequence
  source: /note=“Description of Artificial Sequence Synthetic primer”
tgatgacggtgaaaacctctga

SEQ ID NO: 46 - DNA, 19 bp, Artificial Sequence
  source: /note=“Description of Artificial Sequence Synthetic primer”
cggcatccgcttacagaca

SEQ ID NO: 47 - protein, 6 aa, Artificial Sequence
  source: /note=“Description of Artificial Sequence Synthetic 6xHis tag”
HHHHHH